(19) United States
(12) Patent Application Publication
(10) Pub. No.: US 2020/0206718 A1
(43) Pub. Date: Jul. 2, 2020

(54) ODOR ABSORBING PAINT AND PREPARATION METHOD AND USE THEREOF

(71) Applicant: TIANJIN LANGWEI BAILI TECHNOLOGY CO., LTD., TIANJIN (CN)

(72) Inventor: GUANG LIU, TIANJIN (CN)

(21) Appl. No.: 16/506,163

(22) Filed: Jul. 9, 2019

(30) Foreign Application Priority Data
Dec. 26, 2018 (CN) 201811600955.5

(51) Int. Cl. B01J 20/20; B01D 53/04; B01J 20/10; B01J 20/24; B01J 20/26; B01J 20/30; B01J 20/32; A61L 9/014

(52) U.S. Cl. CPC B01J 20/20; A61L 2209/22; B01J 20/10; B01J 20/24; B01J 20/261; B01J 20/3042; B01J 20/3214; B01J 20/324; A61L 9/014; B01D 2253/25; B01D 2253/102; B01D 2253/106; B01D 2257/93; B01D 2257/90; B01D 53/04

(57) ABSTRACT

A method and system relate to an odor absorbing paint and a preparation method thereof. Some embodiments of the disclosure provide a method for making an odor absorbing paint by binding activated carbon particles to tourmaline particles with acrylic emulsion as an adhesive. In other embodiments, the tourmaline particle component in the odor absorbing paint can release negative ions for a long time. These released negative ions can form neutral coordination compounds with indoor toxic and harmful gas particles. In further embodiments, the coconut shell activated carbon particle component can adsorb the neutral coordination compounds, so that the toxic and harmful gas particles will remain in tubular micropores inside the activated carbon, and will not be desorbed to leave.

### CROSS REFERENCE TO RELATED APPLICATIONS

This application claims priority to Chinese application number 20181160095-5.5 filed on Dec. 26, 2018, the disclosure of which is incorporated by reference herein in its entirety.

### FIELD OF THE DISCLOSURE

The disclosure relates generally to the field of building materials. More specifically, the disclosure relates to the field of an odor absorbing paint, and a preparation method and use thereof.

### BACKGROUND

Indoor toxic and harmful gases: relevant data indicate that indoor air pollution is 5 to 10 times as serious as that outdoors, and there are as many as more than 500 pollutants. Indoor air pollution has become a cause of many diseases. The Chinese national standard GB/T 18883-2002 Indoor Air Quality Standard stipulates to control 15 major toxic and harmful gases causing indoor air pollution and endangering human health. The 15 toxic and harmful gases include: 1. sulfur dioxide: it is a preservative for manufacturing wine and fruit wine, is a brightener for bleaching paper, broken filament, straw hats, dried fruits, vermicelli, and the like, and is released into indoor air when these articles are used; 2. nitrogen dioxide: mainly sourced from fuel combustion, automobile exhaust, lightning, and the like; 3. carbon monoxide: mainly sourced from firing for indoor heating, tea-urn, stove, and the like; 4. carbon dioxide: indoor carbon dioxide is mainly sourced from fuel combustion, cooking, organism release, and the like; 5. ammonia: indoor ammonia gas is mainly sourced from volatilization of substances containing urea or ammonia water in buildings and decorations, decay of indoor organic matters, sewer odor, and the like; 6. ozone: petrochemical combustion is an important pollution source of ozone, and is a photochemical product of irradiating nitrogen oxides by the UV in air, and irradiation of the toner cartridge of a copier also produces ozone; 7. formaldehyde: wood processing, plywood, fiberboard, density board, wood floor, furniture, wall cloth, wallpaper, carpet, paint, paper, clothing, cosmetics, antiseptic, adhesive, and the like; soaking aquatic food in water, whitening fumigated food, and the like; all these products will release formaldehyde, thermal aging and deterioration of foams in decorations, and the like; and even 0.17 mg of formaldehyde can be released when smoking a cigarette. 8. benzene: benzene is volatilized from synthetic dyes, synthetic rubber, synthetic resin, synthetic fiber, synthetic grain, plastics, pharmaceuticals, pesticides, photographic film, varnish, nitrocellulose lacquer, diluent, paint remover, lubricant, grease, wax, celluloid, resin, artificial leather, and the like; 9. toluene: toluene is released from interior decoration, furniture adhesive, paint, coating, wood lacquer, waterproofing material diluent, fuel combustion, smoking, and the like; 10. xylene: xylene is released from interior decoration, furniture adhesive, paint, wallpaper, carpet, pesticide, photocopier, and the like; 11. benzo[a]pyrene: there is high content of benzo[a]pyrene in flue gas arising from combustion of various types of carbon black and coal, petroleum, and the like, cigarette smoke, vehicle exhaust, inappropriate frying in food chain, barbequed food, and fumigated food; 12. inhalable particulate matter (PM10): indoor inhalable particulate matters are mainly sourced from outdoor floating dust, incomplete fuel combustion, lampblack, and the like in indoor kitchen. Inhalable particulate matters will also arise from prolonged aging and abrasion of building insulation materials (asbestos), and fur particles of pets, animals, and poultries diffused indoors. The best living space for human should be 20 m above the ground, and preferably within 10 m. For inhalable particulate matters, the closer are they to the ground, the larger are their diameters, and the smaller is the probability of being inhaled by human. We know that the mass of a toxic gas is very small, it is basically mixed with air, and can be very easily inhaled by human. Then, for us humans, the higher our living space is from the ground, the more harmful effects the inhalable particulate matters have on the human. However, with the human population growth, and increase of the urban population density, the office and residential buildings are built higher and higher, there are row upon row of skyscrapers, and the inhalable particulate matters are increased with the enhanced height of the buildings; 13. total volatile organic compound (TVOC): organic compounds involved in atmospheric photochemical reactions are mainly sourced from: combustion products of fire coal, natural gas, and the like, smog from smoking, heating, cooking, and the like, paint, oil paint, adhesives, wallpaper and other decorations, printing ink, correction fluid, office supplies, furniture, carpet, chemical fiber curtains, and household appliances. Volatile organic compounds will arise from footwear glue, fragrant toys, detergents, cleansers, sprays, fabric softener, cosmetics, human emissions, and the like; 14. radon: indoor radon is mainly sourced from building materials, e.g., granite and other natural stone, brick, sand, cement, plaster, and the like. Radon can pass through cracks between cement floors and walls, ground cracks, small holes on hollow brick walls, basements, cellars or other contact areas in contact with soil, sinkholes, sewers, odor when flushing the toilet, and the like; 15. total bacterial count: bacteria are bred in moisture in building materials, air conditioners, humidifiers, washing machines, vegetable washing sinks, etc., thereby forming collosol spread in air, and endangering food and water; and 16. static electricity: many indoor electrical devices, such as TV set, computers, mobile phones, air conditioners, electric heaters, electric hair dryers, and the like, will release static electricity when they are used. Negative ions in air are consumed, so that there are relatively more positive ions indoors. People uptake more positive ions into their bodies during breathing. Positive ions consist of hydrogen ions, blood and other liquid will tend to be acidic when in vivo hydrogen ion concentration is increased. In vivo acidness hinders smooth metabolism, resulting in cell function decline, accelerated aging, and immunity decrease, and further resulting in various diseases. In short, among these indoor toxic and harmful gases, some are caused by fuel combustion, cooking, smoking, etc., and most of them can be removed by forced ventilation methods, such as range hoods, ventilation fans, and opening windows; while some are emitted from building materials, decoration materials, furniture, carpets, curtains, home appliances, clothes, tools, toys, laundry detergents, and the like all the time. They have different properties, arise in different ways, and do different degrees of harm to people. Some are harmful to human eyes, while some enter human bodies through the skin or respiratory tracts, and harm the human respiratory tracts, internal organs, and blood. Among these toxic and harmful gases, in some circumstances, one toxic and harmful gas produces a plurality of hazards, while in some circumstances, a plurality of toxic and harmful gases produces one superimposed hazard. They are toxic and harmful gas particles, and should be subject to comprehensive treatment, to keep the indoor air fresh.

Some prior art references provide methods for eliminating indoor toxic and harmful gases such as: 1. ventilation method: It takes 2-3 years to reduce the toxicity and unpleasant odor of interior decoration with the ventilation method, additional toxic and harmful gases will be continuously released in the future life, and opening windows for ventilation is affected by air conditioning in summer and heating in winter; 2. plant method: It mainly includes planting flowers and grass for adsorption and absorbing some harmful substances using photosynthesis; the plants will absorb oxygen and release carbon dioxide at night without photosynthesis, and can only absorb very limited amount of harmful substances, which plays a little role; 3. deodorant method: It mainly includes spaying an air freshener, and the unpleasant odor is only masked by a fragrance odor, but actually the toxic and harmful gases are not removed; 4. photoelectric deodoring method: Toxic and harmful substances are eliminated by irradiation using high energy ozone-UV, but the arising UV and ozone are both secondary pollution, and are difficult to be used by households; 5. negative ion generator: The negative ion generator breaks apart an oxygen molecule in air by transmitting high voltage corona, enables one oxygen atom to agglomerate with a positive ion particle of a toxic and harmful gas and then settle, and enables the other oxygen atom to form ozone by binding to an oxygen molecule, thereby causing secondary pollution; and 6. physical adsorption method: It refers to adsorption by placing activated carbon, odor absorbing rods, or the like. This method only adsorbs some toxic and harmful particles into in vivo tubular micropores without changing the toxic and harmful substance itself. When the adsorption reaches a saturation state, desorption will take place, and the toxic and harmful substance adsorbed onto the surface of the activated carbon will be released into the air again. Positive ion particles of toxic and harmful gases in air are neutralized by adding a small amount of negative ion powder in the paint, to form complex sedimentation. Negative ion powder is a synthetic mixture containing rare earth element, and will have radioactive contamination; adsorption using sepiolite: Sepiolite is obtained by alteration of serpentinite, serpentinite is the main component of asbestos, and the tragedy of affecting human health by asbestos can be repeated by no means. Some tourmaline power is added in the paint to purify the air. Since the tourmaline powder is a transparent or semitransparent crystal, the main components of the paint include emulsion, water and titanium dioxide. Among high quality paint components, only when the emulsion content is 50-65%, will the paint film have a sense of delicate skin, the contrast ratio of the hiding power of qualified products is 92%, the contrast ratio of superior products is greater than 96%, and only by adding 15-20% titanium dioxide with hiding power, can the products meet the requirements, which will enable the paint component to reach the proportion of 65-85%. In addition, 15-25% of water is still required to prepare a slurry of titanium dioxide and disperse titanium dioxide, and there is no extra room to add tourmaline powder. Tourmaline powder is transparent or semitransparent crystalline particles without hiding power, and cannot replace much titanium dioxide, which will limit the addition amount of tourmaline powder, and the paint film is compact, thereby seriously hindering tourmaline powder from irradiating negative ions and neutralizing harmful substances, so that it is difficult to achieve the purpose of keeping indoor air purification for a long time using the paint.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

Some exemplary embodiments of the disclosure provide an odor absorbing paint, which helps keep indoor air in a clean state for a long time and achieves comprehensive purification of indoor air.

In some embodiments, the disclosure provides an odor absorbing paint, including components of the following mass percentage: 20-45% coconut shell activated carbon particles, 18-29% water, 12-53% tourmaline particles, 3-6% hydroxyethyl cellulose solution, and 6-8% acrylic emulsion.

Optionally, the mesh number of the coconut shell activated carbon particles is 20-40.

Optionally, the tourmaline particles include 10-20 mesh tourmaline particles and 20-40 mesh tourmaline particles. The mass fraction of the 10-20 mesh tourmaline particles in the odor absorbing paint is 5-13%. The mass fraction of the 20-40 mesh tourmaline particles in the odor absorbing paint is 7-40%.

Optionally, the mass concentration of the hydroxyethyl cellulose solution is 3-6%.

In other embodiments, the disclosure provides a preparation method of the above-described odor absorbing paint, including the following steps. (1) Immersing coconut shell activated carbon particles in water, until reaching saturated water absorption of the coconut shell activated carbon particles, to obtain a water-saturated coconut shell activated carbon particles. (2) Mixing the acrylic emulsion, the water-saturated coconut shell activated carbon particles, the tourmaline particles, and hydroxyethyl cellulose solution, to obtain the odor absorbing paint.

Optionally, the immersion lasts for 24-32 hrs at 5-35° C.

Optionally, the mixing is carried out while stirring, and the stirring is carried out at a rotational speed of 50-80 r/min.

According to an embodiment, the disclosure provides a use of the above-described odor adsorbing paint or the odor absorbing paint prepared by the above-described preparation method in removing indoor toxic and harmful gases.

According to another embodiment, the disclosure provides a use of the above-described odor adsorbing paint or the odor absorbing paint prepared by the above-described preparation method in removing toxic and harmful gases in a confined space.

According to a further embodiment, the disclosure provides a use of the above-described odor adsorbing paint or the odor absorbing paint prepared by the above-described preparation method in a container box.

In further embodiments, the disclosure provides an odor absorbing paint. The disclosure provides a method for making an odor absorbing paint by binding activated carbon particles to tourmaline particles with an acrylic emulsion adhesive. The tourmaline particles in the odor absorbing paint can release negative ions for a long time. Toxicological toxic and harmful gases, such as indoor ammonia gas, ozone, formaldehyde, benzene, toluene, xylene, benzo[a]pyrene, total volatile organic compound (TVOC), and bacteria, are mostly positive ions with positive charge. Thus, negative ions released by the tourmaline particle component in the odor absorbing paint neutralize with indoor toxic and harmful gas particles to form neutral coordination compounds. The coconut shell activated carbon particle component can adsorb the neutral coordination compounds, so that the neutral coordination compound particles are adsorbed to remain in tubular micropores inside the coconut shell activated carbon particle component, and will not be desorbed to leave. The indoor static moiety is positive charge in a static state. The tourmaline particle component in the odor absorbing paint releases negative ions for a long time, and neutralizes with positive electrostatic charge, to eliminate indoor static electricity. For the indoor fine particulate matter moiety, such as sulfur dioxide, nitrogen dioxide, carbon monoxide, carbon dioxide, inhalable particulate matter (PM10), and colony remains, other indoor particulate matters are adsorbed by the coconut shell activated carbon particle component into its own internal tubular micropores except that the fuel combustion, cooking lampblack, and the like are eliminated by forced ventilation, such as range hoods, ventilation fans, and opening windows. Radioactive substance moiety of indoor radon 222Rn is adsorbed by the coconut shell activated carbon particle component into its own internal tubular micropores. By joint coordinated action of the coconut shell activated carbon particle component and the tourmaline particle component in the odor adsorbing paint, toxic gases of the indoor toxic and harmful gas particles are neutralized into the complex compound particle moiety, electrostatic particle moiety, floating dust moiety of particulate matters, and radioactive material particle moiety, which are then completely eliminated. The odor absorbing paint coated indoors helps keep indoor air in a clean state for a long time, and achieve a prolonged comprehensive treatment of indoor toxic and harmful gases.

### DETAILED DESCRIPTION

In some embodiments, the disclosure provides an odor absorbing paint which may include components of the following mass percentage: 20-45% coconut shell activated carbon particles, 18-29% water, 12-53% tourmaline particles, 3-6% hydroxyethyl cellulose solution, and 6-8% acrylic emulsion.

According to an embodiment, the odor absorbing paint provided in the disclosure may include coconut shell activated carbon particles of a 20-45% mass percentage, preferably 30-43%, and more preferably 35-42%. According to an embodiment, the mesh number of the coconut shell activated carbon particles may be 20-40. A large amount of pore size of the coconut shell activated carbon particles selected in the disclosure may be: large pore size 5-10 μm, transitional pore size 5000-5 nm, micropore size <2-5 nm, which are slightly larger than the diameter of the toxic and harmful gas molecule, (PM10 represents particles with the particle size of less than 10 μm), and have very strong adsorption capacity. The selected 20-40 mesh coconut shell activated carbon particles may have a large specific surface area (up to 900-1700 m²/g), have developed internal void, have low density, have both physical adsorption capacity and chemical adsorption capacity, and may adsorb toxic and harmful substances in both gas phase and liquid phase, which may purify an indoor air.

According to another embodiment, the odor absorbing paint provided in the disclosure may include water of a 18-29% mass percentage, preferably 22-28%, and more preferably 24-26%. The disclosure enables the coconut shell activated carbon particles to reach saturated water absorption using water, which may remove ash powders in the coconut shell activated carbon particles, and enables tubular micropores inside the coconut shell activated carbon particles to be prefilled with water and occupy the inner space, so that it is difficult for the acrylic emulsion to enter the tubular micropores inside the coconut shell activated carbon. Furthermore, in the drying and dewatering process after the odor absorbing paint is coated, saturated water pre-filled in the tubular micropores inside the coconut shell activated carbon is evaporated, leaving a passage for water evaporation, and leaving a passage for subsequent adsorption of toxic and harmful gas particles in air.

According to a further embodiment, the odor absorbing paint provided in the disclosure may include tourmaline particles of a 12-53% mass percentage. In the disclosure, the tourmaline particles may include 10-20 mesh tourmaline particles and 20-40 mesh tourmaline particles. In the disclosure, the 10-20 mesh tourmaline particles specifically refer to the over tail obtained by enabling the siftage of a 10 mesh sieve to pass through a 20 mesh sieve, The 20-40 mesh tourmaline particles specifically refers to the over tail obtained by enabling the siftage of a 20 mesh sieve to pass through a 40 mesh sieve. In the disclosure, the mass fraction of the 10-20 mesh tourmaline particles may be 5-13%, and preferably 7-12%, The mass fraction of the 20-40 mesh tourmaline particles in the odor absorbing paint may be 7-40%, and preferably 10-15%. The disclosure enables toxic and harmful gases and negative ions to be released from tourmaline to form coordination compounds using the characteristic of the tourmaline particles capable of releasing negative ions, thus eliminating the polarity and free radicals of the toxic and harmful gases, so that the toxic and harmful gases remain in tubular micropores inside the coconut shell activated carbon particles, and will not be desorbed to leave, to achieve effective removal of indoor toxic and harmful gases. The disclosure may further guarantee, by adding the 10-20 mesh tourmaline particles, that when the odor absorbing paint is coated, the design thickness (3-5 mm) may be achieved by controllable coating of particles of large particle size.

According to one embodiment of the disclosure, some toxic and harmful gases directly coordinate and neutralize with negative ions released from the tourmaline particle. Some toxic and harmful gases may be adsorbed into the coconut shell activated carbon particles, and then coordinate and neutralize with the negative ions released from the tourmaline particles. By controlling the ratio of the tourmaline particles to the coconut shell activated carbon particles, the disclosure may ensure that the negative ions released from the tourmaline particles may not only meet the requirements for coordinating and neutralizing toxic and harmful gases, but also meet the requirements for neutralization with positive electrostatic charge in indoor air. The disclosure may better meet the requirements for coordinating and neutralizing toxic and harmful gases adsorbed into tubular micropores inside the coconut shell activated carbon particles, so that the toxic and harmful gases that have been adsorbed into the tubular micropores inside the activated carbon neutralize and coordinate with the negative ions released from tourmaline to form a complex compound, and will not be desorbed to leave. The coconut shell activated carbon particle component of the disclosure may not only meet the requirements for the capacity of forming neutral coordination compound particles by neutralizing absorbed indoor toxic and harmful gas particles with negative ions released from the tourmaline particle component, but also meet the requirements for the capacity of adsorbing floating dust moiety of fine particles, and may meet the requirements for the capacity of adsorbing radioactive particles, such as radon. The proportional relation between the coconut shell activated carbon particles and the tourmaline particles selected in the disclosure enables both to be uniformly arranged and interdicted from each other, so that the tourmaline particles may be in close contact with the coconut shell activated carbon particles everywhere, which not only helps keep indoor air in a clean state for a long time, but also plays a role in finishing and fireproofing as a sand textured odor absorbing paint.

The odor absorbing paint provided in the disclosure may include hydroxyethyl cellulose solution of a 3-6% mass percentage, and preferably 4-5%. In the disclosure, the mass concentration of the hydroxyethyl cellulose solution may be preferably 3-6%. The disclosure increases workability of the odor absorbing paint using the hydroxyethyl cellulose solution.

The odor absorbing paint provided in the disclosure may include acrylic emulsion of a 6-8% mass percentage, and preferably 6-7%. In the disclosure, during film formation of the acrylic emulsion, after water evaporation, many unsaturated free ends are formed by emulsification and polymerization of acrylate, and linkage may be formed between unsaturated free ends to form a saturated reticular paint film. The disclosure mixes and connects the tourmaline particles with the coconut shell activated carbon particles with the acrylic emulsion as an adhesive. The disclosure controls use amount of the acrylic emulsion, reduces polymerization degree of the acrylic emulsion, and makes it become an intermittent mesh. The disconnected moiety also has a linkage between unsaturated free ends, which may not only guarantee adhesion between the tourmaline particles and the activated carbon particles, guarantee adhesion between the odor adsorbing paint and the substrate, and guarantee that the paint film formed by the acrylic emulsion may completely wrap the tourmaline particles and the activated carbon particles, but also ensure release of negative ions and smoothness of the passage for adsorbing toxic and harmful substances, thereby obtaining a sand textured odor adsorbing paint.

The disclosure provides a preparation method of the odor absorbing paint according to the above technical solution, including the following steps. (1) Immersing the coconut shell activated carbon particles in water, until reaching saturated water absorption of the coconut shell activated carbon particles, to obtain a water-saturated coconut shell activated carbon particle. (2) Mixing the acrylic emulsion, the water-saturated coconut shell activated carbon particles, the tourmaline particles, and hydroxyethyl cellulose solution, to obtain the odor absorbing paint.

The disclosure immerses the coconut shell activated carbon particles in water, until reaching saturated water absorption of the coconut shell activated carbon particles, to obtain a water-saturated coconut shell activated carbon particle. In the disclosure, the immersion lasts preferably at 5-35° C., and more preferably at 15-30° C., preferably for 24-32 hrs, and more preferably for 26-28 hrs. In the disclosure, saturated water absorption of the coconut shell activated carbon particles may be preferably 60-70%, and preferable 65%. The disclosure immerses the coconut shell activated carbon particles in water, which enables tubular micropores inside the activated carbon not to be blocked by ash powders and acrylic emulsion, thus ensuring adsorption capacity of the activated carbon for toxic and harmful gas particles.

After obtaining the water-saturated coconut shell activated carbon particles, the disclosure mixes the acrylic emulsion, the water-saturated coconut shell activated carbon particles, the tourmaline particles, and hydroxyethyl cellulose solution, to obtain the odor absorbing paint. In the disclosure, the mixing may be carried out with stirring, rotational speed for the stirring may be 50-80 r/min, and more preferably 60-70 r/min. The disclosure does not particularly define the mixing approach, and may select an approach well known to those skilled in the art as long as it enables the components to be fully mixed. The disclosure may first mixes the acrylic emulsion, the water-saturated coconut shell activated carbon particles and the tourmaline particles, and then mixes the resulting mixture with the hydroxyethyl cellulose solution, to enable water in the acrylic emulsion, water in the hydroxyethyl cellulose, water in the water-saturated coconut shell activated carbon, and separately added water to be evaporated during film formation of the odor absorbing paint, and to form passages for water evaporation, thereby leaving passages for releasing negative ions by the tourmaline particles, and adsorbing toxic and harmful gas particles by the coconut shell activated carbon particles. The disclosure may first immerse the coconut shell activated carbon in water until reaching saturation, to prevent the acrylic emulsion from permeating into tubular micropores inside the coconut shell activated carbon while mixing by stirring, which affects the adsorption capacity. The disclosure may select low speed stirring at 50-80 r/min, to prevent breaking up the swampy coconut shell activated carbon, which affects porosity of sand textured odor absorbing paint during drying for film formation, and affects release of negative ions inside the paint film and adsorption of toxic and harmful gases.

The disclosure provides a use of the odor absorbing paint in the above technical solution in removing indoor toxic and harmful gases. The “indoor” in the disclosure refers to the “indoor” in the general sense in the art, and may be the “indoor” capable of achieving forced ventilation conditions.

According to the real life habits of people and the generation and sources of 15 major indoor toxic and harmful gases controlled in the national regulations at present, three hypotheses are made: Hypothesis I: supposing that indoor toxic and harmful gases are 30% above the upper limits, doors and windows are opened for ventilation, so that the indoor air quality meets the national quality control standard, while on the next day, new toxic and harmful gases are released, their contents are 30% above the upper limits again, and ventilation may be conducted again to meet the standard, i.e., toxic and harmful gases that are 30% of the amount defined in the national quality control standard are produced indoors every day. According to calculation of 1 m² of indoor area with 3 m clear height, 164.25 mg of sulfur dioxide, 78.84 mg of nitrogen dioxide, 3285 mg of carbon monoxide, 424.75 g of carbon dioxide (based on 1.293 g/L air density), 65.7 mg of ammonia gas, 52.56 mg of ozone, 32.85 mg of formaldehyde, 36.14 mg of benzene, 65.7 mg of toluene, 65.7 mg of xylene, 328.5 mg of benzo[a]pyrene, 49.28 mg of inhalable particulate matter (PM10), and 197.1 mg of total volatile organic compound (TVOC) are produced every year, and 429171.62 mg of toxic and harmful gases are totally generated per m² every year, including 3285 mg of carbon monoxide accounting for 0.76543% of the total amount of the toxic and harmful gases; 424750 mg of carbon dioxide accounting for 98.96973% of the total amount of the toxic and harmful gases, carbon monoxide and carbon dioxide accounting for 99.73516% of the total amount of the toxic and harmful gases; and 11 other toxic and harmful gases produce 1136.62 mg of substances, accounting for 0.26484% of the total amount of the toxic and harmful gases. Furthermore, 131,400 radioactive atoms of Bekele radon 222Rn will decay per m² every year, and 373,800 bacterial colony forming units may be formed. Hypothesis II: in view of the fact that carbon monoxide is an intermediate product mainly arising from combustion of carbonaceous fuels. For example, the indoor carbon monoxide in a household is caused mainly by gas stove or cooking, and people will generally open the exhaust system or windows for ventilation when using the gas stove, which will greatly reduce the remaining amount of carbon monoxide. Carbon dioxide is also caused mainly by fuel combustion and cooking, and a part of carbon dioxide is released from human or organisms. Assuming that the emission amount of carbon monoxide and carbon dioxide caused by fuel combustion, cooking, etc. is emitted outdoors mainly by a forced ventilation system, such as a range hood, or door and window ventilation, and the part of carbon dioxide released from human or organisms is treated together with other indoor toxic and harmful gases. In the Hypothesis II, 90% of carbon monoxide and carbon dioxide are emitted by forced ventilation, 385232 mg is emitted per m² every year, 10% is remaining, and 42804 mg is remaining per m² every year. 43941 mg of toxic and harmful gases are totally produced per m² every year, in which 42804 mg of the remaining carbon monoxide and carbon dioxide account for 97.41% of the totally produced toxic and harmful gases per m² every year, while other 11 toxic and harmful gases account for 2.59%. With 100 m² use area as a converted standard unit, by 50th year, 21.97 kg of toxic and harmful gases may be totally produced. Hypothesis III: The coconut shell activated carbon may at most adsorb other particles less than diameters of micropores inside it until approaching or reaching its own mass. Considering that the negative factors, such as manufacturing, construction, and use, affect the absorption capacity of the activated carbon, and supposing that the activated carbon adsorbs 70% of its own mass, 31.39 kg of the activated carbon is required to adsorb 21.97 kg of toxic and harmful gases per 100 m² in 50 years. In short, the above three hypotheses are as follows: hypothesizing that the activated carbon absorbs toxic and harmful gases that are 30% of the amount defined in the national standard every day in 50 years; hypothesizing that most carbon monoxide and carbon dioxide arising from combustion or cooking are emitted by forced ventilation, such as range hoods and opening windows, and the part of the gases released from human and organisms is absorbed by the activated carbon; and hypothesizing that the coconut shell activated carbon may absorb toxic and harmful gases that are 70% of its own weight. Under the hypothesized conditions, in an indoor standard area unit (300 m3) converted from 100 m² of 3 m storey height in 50 years, 31.39 kg of the coconut shell activated carbon is required to adsorb 21.97 kg of toxic and harmful gas particles, i.e., 31.39 kg of the coconut shell activated carbon is required for the converted standard area unit, 156.95-52.32 kg of the odor absorbing paint is required for the converted 100 m2 standard use area unit, at least 3-5 mm thickness shall be coated, and 8.38-41.85 m² of the odor adsorbing paint may be coated for finishing (hypothesizing that the volume weight of the odor absorbing paint is 1250 kg/m3). By the above hypotheses and quantitative calculation, quantitative use of the odor absorbing paint in removing indoor toxic and harmful gases is achieved.

Based on the hypotheses, total mass of the indoor toxic and harmful gas particles may be quantified to provide the total requisite amount of the tourmaline particle component to release negative ions, and provide the activated carbon particle component to adsorb toxic and harmful gas particles in certain time, to ensure the total amount of the toxic and harmful gas particles, namely indoor toxic particles, floating dust particles, electrostatic particles, and radioactive particles, capture and adsorption of which are completed under the hypothesized conditions in certain length of time. The disclosure achieves determining total use amount of the activated carbon particle component, and total use amount of the odor absorbing paint in a unit area based on the total mass of the toxic and harmful gas particles to be adsorbed under the hypothesized conditions within certain length of time, and then sets use amount of the odor absorbing paint in each subarea according to the subareas of a relatively confined space in the unit area, implements coating, to achieve the purpose of quantitative comprehensive indoor air purification.

The disclosure provides a use of the odor absorbing paint in the above technical solution in removing toxic and harmful gases in a confined space. The confined space in the disclosure specifically refers to a space where forced ventilation cannot be achieved. The disclosure uses the odor absorbing paint in a confined space, and may achieve adsorbing indoor toxic and harmful gas particles in a confined space by increasing the amount of the odor absorbing paint or reducing the service life of the odor absorbing paint, such as use in a confined space, e.g., basements, tunnels, vehicles, ships, and submarines, for air purification.

The disclosure provides a use of the odor absorbing paint in the above technical solution in a container box.

The odor absorbing paint of the disclosure may be coated on other materials, such as a sheet material, and canvas, e.g., putting in a ventilating container box for absorbing toxic and harmful gas particles and purifying air as a flexibly used product.

The odor absorbing paint provided in the disclosure may be implemented indoors in high rise: generally in high-rise buildings higher than 20 m, indoor dust particles having a particle diameter of PM10 or less, especially dust particles having a particle diameter of PM2 or less may be increased with the increase of the height. The higher is the height, the more and the smaller are the dust particles, and the greater are they harmful to human. Therefore, with the increase of the building height, the use amount of the indoor odor absorbing paint may be increased in accordance with the regulation factor. For example, when the building height is increased by 20 m, the use amount of the indoor odor absorbing paint is increased by 10%.

Some colored particulate substances, e.g., useful materials such as giant clam particles, may be added to the odor absorbing paint provided in the disclosure according to the requirements of the practical use of color, which may increase the effects of tranquilizing mind, improving insomnia, and keeping fitness, and may further adjust color. Harmless materials, such as white marble, natural color sand particles, and the like, may also be added to adjust the color, which, however, shall not affect the use amount of the activated carbon per indoor unit area and the ratio of activated carbon to tourmaline. Moreover, after color particles are newly added, radioactive granite particles, hornblende particles, negative ion powder, or the like, sepiolite or other asbestos-containing particles cannot be added, nor may new pollution be caused. Diatom ooze, shell powder, or other powder materials, or dust materials cannot be added, nor may tubular micropores inside the activated carbon be blocked.

The odor absorbing paint and the preparation method thereof as provided by the disclosure are further described below in conjunction with the following embodiments, which should not be construed as limiting the extent of protection of the disclosure.

### EMBODIMENT 1

An odor absorbing paint was prepared from the following components: 7% acrylic emulsion, 40% 20-40 mesh coconut shell activated carbon particles, 26% water, 10% 10-20 mesh tourmaline particles, 13% 20-40 mesh tourmaline particles, and 4% hydroxyethyl cellulose solution of the mass concentration of 4%. Immersing the coconut shell activated carbon particles in water at 25° C. for 28 hrs, until reaching saturated water absorption of the coconut shell activated carbon particles, to obtain a water-saturated coconut shell activated carbon particle. Fully mixing the acrylic emulsion, the water-saturated coconut shell activated carbon particles, and the tourmaline particles at 60 r/min, adding the hydroxyethyl cellulose solution to the resulting mixture, fully mixing, testing the product, filling, packaging, warehousing or delivering the product, and labeling volume weight and activated carbon content, to obtain the odor absorbing paint.

### EMBODIMENT 2

An odor absorbing paint was prepared from the following components: 6% acrylic emulsion, 20% coconut shell activated carbon particles, 13% water, 5% water besides saturated water of the activated carbon, (an appropriate amount of water shall be added when the proportion of dry tourmaline particles may be large, to obtain a product of a suitable workability), 13% tourmaline particles (10-20 mesh), 40% tourmaline particles (20-40 mesh), and 3% hydroxyethyl cellulose solution. Immersing the coconut shell activated carbon particles in water at 30° C. for 24 hrs, until reaching saturated water absorption of the coconut shell activated carbon particles, to obtain a water-saturated coconut shell activated carbon particle. Successively adding the water-saturated coconut shell activated carbon particles, the tourmaline particles, 5% water, and the acrylic emulsion in a dispersor, fully mixing at 70 r/min, adding the hydroxyethyl cellulose solution to the resulting mixture, fully mixing, testing the product, filling, packaging, warehousing or delivering the product, and labeling volume weight and activated carbon content, to obtain the odor absorbing paint.

### EMBODIMENT 3

An odor absorbing paint was prepared from the following components: 8% acrylic emulsion, 45% coconut shell activated carbon particles, 29% water, 5% tourmaline particles (10-20 mesh), 7% tourmaline particles (20-40 mesh), and 6% hydroxyethyl cellulose solution. Immersing the coconut shell activated carbon particles in water at 20° C. for 32 hrs, until reaching saturated water absorption of the coconut shell activated carbon particles, to obtain a water-saturated coconut shell activated carbon particle. Fully mixing the acrylic emulsion, the water-saturated coconut shell activated carbon particles, and the tourmaline particles at 80 r/min, adding the hydroxyethyl cellulose solution to the resulting mixture, fully mixing, testing the product, filling, packaging, warehousing or delivering the product, and labeling volume weight and activated carbon content, to obtain the odor absorbing paint.

The odor absorbing paint prepared in Embodiments 1 to 3 may be used for purifying indoor air. When the odor absorbing paint absorbs indoor toxic and harmful gas particles, the odor absorbing paint may be required to have certain coating area, to ensure that there is certain contact area between the odor absorbing paint finishing and air, enabling tourmaline to fully release negative ions and neutralize indoor toxic and harmful gases, so that the activated carbon particles have enough area and smooth passages for adsorbing toxic and harmful gas particles, or adsorbing coordination compound particles obtained from neutralization by negative ions having been released by tourmaline. In the above embodiments, the calculation may be made under three hypothesized conditions: the standard area unit may be converted from 100 m² of 3 meter storey height in 50 years, 31.39 kg of the coconut shell activated carbon may be required to adsorb 21.97 kg of toxic and harmful gas particles, and if the effective service life may be reduced to 25 years, and the coating thickness may be decreased from original 4 mm to 2 mm, the use amount of the activated carbon required in 25 years and the coating area are guaranteed to remain unchanged. Further reduction of the effective service life, and reduction of the coating area will affect the contact area between the finishing and air, and affect release of negative ions by tourmaline and adsorption of toxic and harmful gases by the coconut shell activated carbon. Reduction of the coating thickness may be bound to reduce the particle size of the coconut shell activated carbon and tourmaline particle. Reduction of the particle size will increase the use amount of the acrylic emulsion, due to the requirements for the paint film strength of the odor absorbing paint, enabling the ventilation capacity of housing with the odor absorbing paint finishing to be reduced. The adsorption capacity of the odor absorbing paint will decrease if the coating film may be very thin.

Some exemplary embodiments of the disclosure provide an odor absorbing paint, and a preparation method and use thereof, and obtains the odor absorbing paint by binding activated carbon particles to tourmaline particles with acrylic emulsion as an adhesive. According to the practical situation and the existing materials, the disclosure clearly indicates toxic and harmful gas particles to be eliminated by forced ventilation and toxic and harmful gas particles to be adsorbed by the coconut shell activated carbon through calculation based on hypothesized conditions: certain converted area, certain service life, and total amount of produced toxic and harmful gas particles, to provide enough absorbent materials and guarantee adsorption, thereby achieving the purpose of comprehensive and quantitative indoor air purification.

Various embodiments of the disclosure may have one or more of the following effects. The indoor coating of the odor absorbing paint may keep an indoor air in a clean state for a long time, achieve a comprehensive treatment of indoor toxic and harmful gases, and quantifying and/or purifying an indoor air. The odor absorbing paint may provide a comprehensive treatment of indoor toxic and harmful gases. The odor absorbing paint may adsorb and eliminate many odorless toxic and harmful gases or gas particles such as carbon monoxide, carbon dioxide, formaldehyde, and radon. The odor absorbing paint may adsorb and eliminate static electricity.

The foregoing descriptions are merely preferred implementations of the present invention. It should be pointed out that for a person of ordinary skills in the art, several improvements and modifications may further be made without departing from the principle of the present invention, and the improvements and modifications should also be considered to fall within the extent of protection of the present invention

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. An odor absorbing paint, comprising:
20-45 wt % coconut shell activated carbon particles;
18-29 wt % water;
12-53 wt % tourmaline particles;
3-6 wt % hydroxyethyl cellulose solution; and
6-8 wt % acrylic emulsion.

2. The odor absorbing paint according to claim 1, wherein a mesh number of the coconut shell activated carbon particles is 20-40.

3. The odor absorbing paint according to claim 1, wherein:
the tourmaline particles comprise 10-20 mesh tourmaline particles and 20-40 mesh tourmaline particles;
the odor absorbing paint comprises 5-13 wt % 10-20 mesh tourmaline particles; and
the odor absorbing paint comprises 7-40 wt % 20-40 mesh tourmaline particles.

4. The odor absorbing paint according to claim 1, wherein a mass concentration of the hydroxyethyl cellulose solution is 3-6%.

5. A method for preparing an odor absorbing paint, comprising the steps of:
immersing coconut shell activated carbon particles in water, until reaching saturated water absorption of the coconut shell activated carbon particles, to obtain water-saturated coconut shell activated carbon particles; and
mixing an acrylic emulsion, the water-saturated coconut shell activated carbon particles, tourmaline particles, and a hydroxyethyl cellulose solution, to obtain the odor absorbing paint;
wherein:
the odor absorbing paint comprises:
20-45 wt % coconut shell activated carbon particles;
18-29 wt % water;
12-53 wt % tourmaline particles;
3-6 wt % hydroxyethyl cellulose solution; and
6-8 wt % acrylic emulsion.

6. The method according to claim 5, wherein the immersing lasts for 24-32 hrs at 5-35° C.

7. The method according to claim 5, wherein the mixing is carried out while stirring, and the stirring is carried out at a rotational speed of 50-80 r/min.

8. A method for removing indoor toxic and harmful gases, comprising the step of using an odor adsorbing paint comprising:
20-45 wt % coconut shell activated carbon particles;
18-29 wt % water;
12-53 wt % tourmaline particles;
3-6 wt % hydroxyethyl cellulose solution; and
6-8 wt % acrylic emulsion.

9. The method according to claim 8, wherein the indoor toxic and harmful gases are in a confined space.

10. The method according to claim 8, wherein the indoor toxic and harmful gases are in a container box.
